(19) United States
(12) Patent Application Publication
(10) Pub. No.: US 2019/0037903 A1
(43) Pub. Date: Feb. 7, 2019

(54) High caloric, high protein nutritional formula comprising collagen

(71) Applicant: Fresenius Kabi Deutschland GmbH, Bad Homburg (DE)

(72) Inventors: Edmundo BRITO DE LA FUENTE, Friedrichsdorf (DE); Susanne KEIM, Bad Homburg (DE); Ericka Pestana, Bad Homburg (DE); Jose Maria Mainou-Sierra, Ober-Moerlen (DE); Maria Femanda MARTINEZ-BOCK, Frankfurt am Main (DE); Eileen WALLENFELS, Rabenau (DE); Susanne Wiegand, Frankfurt Am Main (DE)

(73) Assignee: Fresenius Kabi Deutschland GmbH, Bad Homburg (DE)

(21) Appl. No.: 16/075,294

(22) PCT Filed: Feb. 3, 2017
(86) PCT No.: PCT/EP2017/052428

§ 371 Date: Aug. 3, 2018

(30) Foreign Application Priority Data
Feb. 3, 2016 (EP) 16154090.1
Aug. 18, 2016 (EP) 16184825.4

(51) Int. Cl. A23L 33/18; A23J 1/20; A23J 3/34; A23L 33/19; A23L 33/00; A61K 38/01

(52) U.S. Cl. CPC A23L 33/18; A23J 1/20; A23J 3/342; A23L 33/19; A23L 33/40; A61K 38/014; A23V 2300/26; A23V 2002/00; A23V 2200/33; A23V 2250/5422; A23V 2250/5424; A23V 2250/55; A61K 38/018

(57) ABSTRACT

A nutritional composition comprises a protein component, a lipid component and a carbohydrate component, minerals, vitamins and water, wherein the protein component comprises at least two different protein sources; wherein the first protein source is hydrolysed collagen; the lipid component provides at least 30 EN % of the total energy of the composition and the first source of protein represents 35-95 wt % based on the total weight of the protein component. The nutritional composition provides a homogeneous emulsion of high energy density (e.g. at least 3 kcal/mL) and high protein density (e.g. at least 14 g/100 mL).

### FIELD OF THE INVENTION

The present disclosure relates to ready-to-use nutritional compositions of high caloric density and high protein content. The compositions herein further comprise a lipid component, a carbohydrate component, vitamins, minerals, water and, preferably, are nutritionally complete. The present disclosure further relates to a process for producing such a composition. Such nutritional compositions are suitable to provide homogeneous nutritionally complete emulsions even at very high energy densities (e.g. at least 2.5 kcal/mL, preferably at least 3 kcal/mL) in combination with very high protein densities (e.g. at least 12 wt %, preferably at least 14 wt %) based on the total weight of the composition).

### BACKGROUND OF THE INVENTION

There is a general need for nutritional compositions providing a high amount of calories and nutrients in a relatively low volume of formula.

Such nutritional compositions are particularly needed for elderly patients having reduced appetite, for patients with reduced tolerance to high or even normal amounts of food, patients in need of more than normal calorie and nutrient supply (e.g. patients suffering from (chronic) wasting diseases).

One issue in feeding patients at risk of or with malnutrition is low compliance with the nutritional therapy. Patients often do not tolerate high or even normal volumes of food and/or nutritional compositions.

Typically, the need for a high amount of calories in relatively low volume is met by increasing the amount of lipid and/or carbohydrate in nutritional compositions. Increasing the protein content is difficult. In particular in compositions comprising minerals and vitamins, such as nutritionally complete compositions, interactions between minerals and proteins make it often impossible to obtain a stable, homogeneous composition.

However, there is often a medical need for compositions being high in protein and calories in a low volume of formula. And still, for mainly technical reasons, nutritional compositions having a high amount of protein typically have a lower caloric density. Also, compositions high in protein are often devoid of or at least low in lipids and/or carbohydrates.

Thus, the protein density of a nutritional composition is typically not simultaneously increased with the caloric density as such an increase typically poses problems in terms of processability, homogenization, sterilization, fouling, instable emulsions and/or high viscosity. These issues are most pronounced for nutritionally complete compositions, i.e. compositions providing minerals and vitamins in amounts rendering them suitable as sole source of nutrition.

Therefore, when it comes to ready to use nutritional compositions and in particular nutritionally complete compositions, the patient/caregiver typically has to choose one of two options: 1. high caloric nutrition or 2. nutrition high in protein.

Accordingly, there is a need for nutritional compositions simultaneously providing a high caloric density and a high amount of protein. In particular there is a need for such a composition being additionally adaptable to be nutritionally complete.

### SUMMARY OF THE INVENTION

The inventors found that the use of a protein component comprising at least two different protein sources, wherein collagen hydrolysate is the first protein source and present at least by 35 wt %, preferably 45 wt % based on the total weight of the protein component, it is possible to provide homogeneous nutritional compositions having both: high caloric density and a high amount of protein in a low volume.

Moreover, the inventors found that such nutritional compositions can be supplemented with minerals and vitamins in sufficient amounts to render them nutritionally complete and still be stable and homogeneous.

In a first aspect, the present disclosure relates to nutritional compositions comprising a protein component, a lipid component, a carbohydrate component, minerals, vitamins and water, wherein the protein component comprises at least two different protein sources; wherein the first protein source is hydrolysed collagen; the lipid component provides at least 30 EN % of the total energy of the composition and the first source of protein represents at least 35 wt %, preferably 45-95 wt % based on the total weight of the protein component.

In a second aspect, the present disclosure relates to a process for making such a nutritional composition.

In a third aspect, the present disclosure relates to the use of such a composition.

In a fourth aspect, the present disclosure relates to a dose unit comprising the nutritional composition.

In a fifth aspect, the present disclosure relates to a dosage regime for applying the nutritional composition.

### DETAILED DESCRIPTION OF THE INVENTION

### Definitions

“High protein” as used herein refers to nutritional compositions wherein the protein component provides at least 15 EN %, preferably at least 18 EN %, most preferred at least 20 EN % based on the total energy of the nutritional composition. In preferred embodiments, such nutritional compositions comprise at least 10 wt %, preferably at least 112 wt %, more preferably at least 14 wt % of protein based on the total weight of the composition. According to the present disclosure, there may be an upper limit to protein content such as at most 40 EN %, preferably at most 30 EN %, more preferred at most 28EN %.

“High caloric” as used herein refers to nutritional compositions having a caloric density of at least 2 kcal/mL, preferably at least 2.5 kcal/ml, more preferably at least 2.8 kcal/mL, most preferably at least 3.0 kcal/mL.

“Hydrolysed collagen” or “collagen hydrolysate” as used herein refers to low molecular weight collagen obtainable by hydrolysis of collagen by procedures known to the skilled artisan. Embodiments of hydrolysed collagen as preferred herein have an average molecular weight M_{w} of from 1000 Da to 6000 Da, preferably 1000 to 3000 Da. Methods for determining the average molecular weight are well known in the art. An exemplary method is given hereinbelow.

“Nutritional composition” herein refers to a synthetically produced food composition. Thus, nutritional compositions are artificial nutritional products obtained by mixing/dissolving bulk ingredients whereby said ingredients are typically provided in solid form (e.g. powders) or liquid from (e.g. oils, water, syrup). The term “nutritional composition” excludes “food”, i.e. non-modified natural food products, such as meat, vegetables, fruits in their natural form and conventionally prepared (e.g. cooked) meals or drinks like tea, coffee or juices.

For the present disclosure, “nutritional compositions” are limited to liquid or semi-solid compositions.

“Patient nutrition” as used herein refers to nutrition intended for individuals suffering from a medical condition. Patient nutrition as defined herein excludes the provision of nutrients in the form of conventionally prepared meals (“food”). Patient nutrition therefore only refers to the provision of nutrients in form of nutritional compositions as defined above. Herein, patient nutrition is intended for patients having high caloric needs and high protein needs.

“Nutritionally complete” refers to nutritional compositions suitable as sole source of nutrition. To be nutritionally complete, it is required that a nutritional composition comprises—in addition to the lipid, carbohydrate and protein components—minerals and vitamins. In order to be nutritionally complete, vitamins and minerals should be present in sufficient amounts as known to the man skilled in the art, i.e. in accordance with established nutritional guidelines. The recommended nutrient requirements, in particular with respect to minerals and vitamins, can be found in standard nutritional guidelines such as EU commission directive 1999/21/EC (see table 1 hereinbelow). Suitable nutrients according to the present disclosure fulfil the requirements of/are listed in REGULATION (EU) No 609/2013.

“Malnutrition” as used herein refers to one or both of Option I: body mass index (BMI, kg/m2)<18.5; Option II: the combined finding of unintentional weight loss (mandatory) and at least one of either reduced BMI or a low fat free mass index (FFMI). Weight loss is defined as either >10% of habitual weight indefinite of time, or >5% over 3 months. Reduced BMI is <20 or <22 kg/m2 in subjects younger and older than 70 years, respectively. Low FFMI is <15 and <17 kg/m2 in females and males, respectively.

A composition “consisting of” a number of ingredients or components is to be understood as comprising no other than the named ingredients or components. In case ranges for amounts of ingredients or components are given, the individual amount of all ingredients or components within the composition has of course also to be adapted such that the sum of all amounts of all present ingredients or components adds up to 100 wt %.

“Homogenization” as used herein refers to the process of diminishing the size of the fat globules in a nutritional emulsion. A preferred homogenization process herein comprises two homogenization steps. The first homogenization step may be carried out at a pressure of 100 bar and the second homogenization step may be carried out at 50 bar. Both steps may be carried out at a temperature of 60-80° C., such as 65-75° C., for example 65° C.

“UHT-treatment” aims at killing of microorganisms. Preferred UHT treatment may be carried out with a pre-heat treatment at 90° C. for 3 min followed by UHT at 139° C. for 6 seconds, followed by a (third) homogenization step requiring homogenization at less than 90° C. with a pressure that can oscillate between 40-150 bar.

“Protein component” as used herein refers to the entirety of ingredients of the nutritional composition declarable as “protein”.

“Lipid component” as used herein refers to the entirety of ingredients of the nutritional composition declarable as “fat”.

“Carbohydrate component” as used herein refers to the entirety of ingredients of the nutritional composition declarable as “carbohydrate”.

“EN %” refers to the contribution of a certain component or of a specific ingredient to the total nutritional energy of an edible composition, e. g the nutritional composition.

“Ready-to-use” refers to the final form of the nutritional composition as administered to a patient. Typically, the nutritional compositions herein are pre-packed in a ready to use format. I.e. sold in separately packed dose units that do not require any further dilution etc.

### Nutritional Compositions

The nutritional compositions herein comprise nutrients in predetermined and controllable amounts. A nutritional composition according to the present disclosure comprises a protein component, a lipid component, a carbohydrate component, minerals, vitamins and water. Optionally, such a nutritional composition may further comprise dietary fibres and/or further ingredients known as food additives.

The nutritional compositions herein typically are liquid or semi-solid and will typically be provided in an oil-in-water emulsion (O/W).

The nutritional compositions are preferably adapted to have a high caloric density of at least 2 kcal/mL, preferably at least 2.5 kcal/ml, more preferably at least 2.8 kcal/mL, most preferably at least 3.0 kcal/mL. Typically, the nutritional compositions will have a caloric density of at most 5.0 kcal/mL, preferably at most 4 kcal/ml, most preferred at most 3.8 kcal/mL. For example, the compositions herein have a caloric density of 3.0-4.0 kcal/mL.

The nutritional composition herein may comprise a lipid component, a protein component, a carbohydrate component, wherein
a. the protein component provides at least 15 EN %, preferably at least 18 EN %, more preferably at least 20 EN % of the total energy of the composition
b. the lipid component provides at least 30 EN % of the total energy of the composition;
c. the carbohydrate component provides at least 20 EN % of the total energy of the composition;

Preferably, in the nutritional compositions according to the present disclosure comprises a lipid component, a protein component, a carbohydrate component, wherein
a. the protein component provides 15-25 EN %, preferably 18-22 EN %;
b. the lipid component provides 40-50 EN %, preferably 43-47 EN %; and
c. the carbohydrate component provides 30-40 EN % preferably 33-37 EN %.

In preferred embodiments, the amount of water comprised in the present composition represents 40-60 wt % based on the total weight of the nutritional composition, preferably 45-55 wt %.

The nutritional composition of the present disclosure is administered enterally, preferably orally.

### Protein Component

The protein component comprises at least two different protein sources, wherein the first protein source is hydrolysed collagen and represents 35 wt %, preferably 45-95 wt %, more preferably 55-90 wt %, even more preferably 70-90 wt %, most preferred 80-85 wt % based on the total weight of the protein component.

Comparing nutritional compositions comprising high amounts of hydrolysed protein the present nutritional compositions (comprising hydrolysed collagen) should lead to improved patient compliance due to improved rheological and sensorial properties like viscosity, texture and/or taste.

The second protein source may be selected from vegetable proteins, animal proteins other than collagen and mixtures thereof. For example milk protein, soy protein, pea protein, egg white and hydrolysates thereof. In preferred embodiments, the second protein source is selected from milk proteins such as total milk protein, milk protein isolate, milk protein concentrate, whey, casein and mixtures thereof. A particularly preferred second protein source is milk protein, e.g total milk protein and/or milk protein concentrate.

Within the second protein source, proteins having different average molecular weights may be used. Preferred average molecular weights (M_{w}) of the proteins used within the second protein source lie in the range of 20-60 kDa. In such a range properties of the nutritional composition can be well balanced in terms of heat stability and/or viscosity.

For example, a second protein source comprising a high amount of a protein having a lower molecular weight will lead to a reduced viscosity of the nutritional composition. Therefore, a preferred second protein source comprises more than 40 wt %, preferably more than 50 wt % of a protein having an average molecular weight of less than 40 kDa, such as 20-40 kDa (based on the total weight of the second protein source).

A preferred protein component comprises collagen hydrolysate as the first protein source and milk protein as the second protein source. Such a protein component is particularly suitable as it can be adapted such that it provides an amino acid distribution suitable to meet current international recommendations for daily intake (e.g. when the nutritional compositions of the present disclosure are used as sole source of nutrition), even without addition of free amino acids, di- or tri-peptides. Such an exemplary international recommendation has been published by the WHO (Technical Report Series 935, 2007, p. 150). Particularly preferred is a protein component comprising 70-90 wt % of hydrolysed collagen as the first protein source and 30-10 wt % milk protein as the second protein source, such as 80 wt % hydrolysed collagen with 20 wt % milk protein (each based on the total weight of the protein component).

Further amino acids may as well contribute to the second protein source. These may be added in their chemical form or in the form of low molecular peptides, such as di- or tri-peptides. However, in preferred embodiments neither free amino acids nor di- or tri peptides are added to the protein component described herein.

Nutritional compositions herein typically comprise at least 10 wt %, preferably at least 12 wt %, more preferably at least 14 wt % of protein based on the total weight of the nutritional composition.

In preferred embodiments, the protein component provides at least 15 EN %, preferably at least 18 EN %, more preferably at least 20 EN % based on the total energy of the nutritional composition. For example the protein component provides 15-25 EN %, preferably 18-22 EN % of the total energy of the composition.

Preferably, the protein to water ratio of the present nutritional composition is at least 2.0/10 [g/g], preferably at least 2.5/10 [g/g].

### Collagen Hydrolysate

As described above, the collagen hydrolysate herein has an average molecular weight M_{w} of from 1000 Da to 6000 Da, preferably 1000 to 3000 Da. Examples are known to the man skilled in the art and commercially available (e.g. via Gelita, Germany). Such hydrolysates and methods for making them are for example described in DE 102010060564 A1, in particular [0004]-[0011] and Example 1 with the low molecular weight hydrolysates in par. [0030] being particularly suitable. Of course, other sources than procine gelating can be used, with bovine being particularly preferred for the applications herein.

Accordingly, particularly preferred collagen hydrolysates have molecular weight distributions listed below:

| | |
|---|---|
| Mol. weight range | Upper limit |

| | | |
|---|---|---|
| >7500 | Da | 10 wt % |
| >3500-7500 | Da | 35 wt % |
| >1500-3500 | Da | 35 wt % |
| >500-1500 | Da | 50 wt % |
| <500 | Da | 15 wt % |

| Mol. weight | | Preferred | | Particularly | | | | | |
|---|---|---|---|---|---|---|---|---|---|
| range | | range | | preferred range | | Example 1 | | Example 2 | |
| >7500 | Da | ≤10 | wt % | ≤5 | wt % | ≤5 | wt % | ≤5 | wt % |
| >3500-7500 | Da | 10-35 | wt % | 10-20 | wt % | 10-20 | wt % | 12-18 | wt % |
| >1500-3500 | Da | 20-35 | wt % | 25-32 | wt % | 25-32 | wt % | 25-31 | wt % |
| >500-1500 | Da | 30-50 | wt % | 40-50 | wt % | 40-50 | wt % | 40-46 | wt % |
| <500 | Da | ≤15 | wt % | ≤15 | wt % | ≤15 | wt % | 5-10 | wt % |

Preferably, at most 10 wt %, more preferably at most 5 wt % of the collagen hydrolysate has a molecular weight of above 7,500 Da.

Preferably, at most 15 wt %, more preferably at most 5 wt % of the collagen hydrolysate has a molecular weight of below 500 Da.

Thus, particularly preferred collagen hydrolysates are characterized by at least 75 wt %, preferably at least 90 wt % falling into the molecular weight range of 500-7500 Da.

### Carbohydrate Component

The carbohydrate component may comprise one or more carbohydrate sources. Typical carbohydrate sources may be selected from the list consisting of maltodextrine, glucose syrup, sucrose, fructose, isomaltulose, starch (modified or unmodified), tapioca dextrine, and mixtures thereof.

Typically, in nutritional compositions herein the carbohydrate component provides at least 25 EN %, preferably 30-40 EN %, for example 33-37 EN % based on the total energy of the nutritional composition.

A preferred carbohydrate component comprises glucose syrup and, preferably sucrose. The carbohydrate component may comprise 65-95 wt % of glucose syrup and 35-95 wt % of sucrose based on the total weight of the carbohydrate component. Preferably, the carbohydrate component comprises 60-80 wt % glucose syrup and 20-40 wt % sucrose, for example 65-75 wt % glucose syrup and 25-35 wt % sucrose based on the total weight of the carbohydrate component.

### Fibre

The nutritional composition herein may comprise ingredients declarable as dietary fibres. Suitable dietary fibres may be selected from the group consisting of cocoa powder, inulin, wheat dextrine, cellulose, microcrystalline cellulose, soy polysaccharides, tapioca dextrine, xanthan, fructooligosaccharides, galactooligosaccharides, at least partially hydrolysed guar gum, acacia gum, pectin, oat fibre, poly dextrose, resistant starch, hemicellulose and mixtures thereof.

### Lipid Component

The lipid component may comprise one or more lipid sources, such as lipids of animal and/or vegetable origin. Suitable lipid sources may be selected from oil of marine origin vegetable oils and combinations thereof. Preferably, lipid sources may be selected from fish oil, sunflower oil, safflower oil, soy oil, rapeseed oil, canola oil, linseed oil and combinations thereof. Additionally, the lipid component may comprise MCT in oil or fat form providing C6-C12 fatty acids.

In terms of individual fatty acids, the lipid component typically includes polyunsaturated fatty acids, monounsaturated fatty acids and saturated fatty acids. Suitable fatty acids may be selected from the group consisting of caproic acid (C6:0), caprylic acid (C8:0), capric acid (C10:0), lauric acid (C12:0), myristic acid (C14:0), palmitic acid (C16:0), palmitoleic acid (C16:1w7), stearic acid (C18:0), oleic acid (C18:1w9), linoleic acid (C18:2w6), a-Linolenic acid (C18:3w3), eicosapentaenoic acid (C20:5w3), docosahexaenoic acid (C22:6w3) and mixtures thereof.

The lipid component provides at least 30 EN %, preferably at least 40 EN % of the total energy of the nutritional composition herein.

In preferred embodiments, the lipid component provides at most 50 EN %.

Preferably, the lipid component provides 40-50 EN %, for example 43-47 EN % based on the total energy of the nutritional composition.

### Vitamins and Minerals

To be regarded as nutritionally complete, nutritional compositions have to comprise vitamins and minerals.

Suitable vitamins to be included in the composition in order to render it nutritionally complete according to the present disclosure are Vitamin A, Vitamin D, Vitamin K, Vitamin C, Thiamin, Riboflavin, Vitamin B6, Niacin, Folic acid, Vitamin B12, Pantothenic acid, Biotin and Vitamin E. An example for rendering a nutritional composition complete in vitamins is given in table 2.

Suitable minerals to be included in the composition in order to render it nutritionally complete according to the present disclosure are Sodium, Chloride, Potassium, Calcium, Phosphorus, Magnesium, Iron, Zinc, Copper, Iodine, Selenium, Manganese, Chromium and Molybdenum. Optionally, Fluoride may be included. An example for rendering a nutritional composition complete in minerals is given in table 3.

### Additives

Nutritional compositions optionally comprise food additives. Additives are typically present in total amount of less than 10 wt %, 5 wt % or even less than 1 wt % based on the total weight of the nutritional composition. Exemplary additives are choline, beta-carotene, lutein, lycopene, caffeine, lecithin, taurine, carnitine, myo-inositol, colorants, aroma and mixtures thereof. Aromas may be caramel, vanilla, yoghurt, chocolate, coffee, cappuccino, fruit aromas and the like.

The additives may include the stabilisers and emulsifiers. Preferably, the stabilisers are selected from gums and mixtures thereof. For example microcrystalline cellulose (E460), sodium carboxymethylcellulose (E466), carrageenan (E407), diacteyl tartaric acid ester of glycerides, cellulose gel (cellulose, microcrystalline). The emulsifiers may be selected from (destilled) monoglycerides such as E471, soy lecithins For example, stabilizers and emulsifiers are included in the following amounts/ratios monoglycerides (E471, as an emulsifier) 1-5 g/L and a stabilizer mixture comprising 0.3-5 g/L, soy lecithin 1-5 g/L, diacetly tartaric acid of glycerides (E472, eg. DATEM) 0.1-5 g/L and MCC 1-8 g/L.

### Use in Therapy

The nutritional composition of the present disclosure may be used in therapy.

For example, the nutritional compositions of the present disclosure may be used in nutritional therapy. In other words, the nutritional composition herein may be used as patient nutrition.

In one embodiment, the nutritional compositions of the present disclosure are for use in prevention and treatment of nutrient deficiencies associated with malnutrition.

In a further embodiment the nutritional compositions of the present disclosure are for use in prevention and treatment of nutrient deficiencies associated with therapeutic fluid restriction. For example, patients requiring therapeutic fluid restriction may be selected from dialysis patients, patients with cardiac insufficiency and patients with liver insufficiency.

In a further embodiment the nutritional compositions of the present disclosure are for use in prevention and treatment of nutrient deficiencies associated with (chronic) wasting diseases such as catabolism, cachexia, including cancer cachexia or cardiac cachexia, HIV, chronic wounds or sarcopenia. The nutritional compositions of the present disclosure are for use in prevention and treatment of nutrient deficiencies in any condition associated with increased protein needs or overall energy, preferably both. Examples are use in prevention and treatment of nutrient deficiencies in gastrointestinal conditions associated with protein and/or energy wasting. Other examples are use in prevention and treatment of nutrient deficiencies of surgical patients, use in prevention and treatment of nutrient deficiencies associated with wounds and pressure ulcers or use in prevention and treatment of nutrient deficiencies associated with cancers.

In one embodiment, the nutritional compositions of the present disclosure are for use in prevention and treatment of protein-energy deficiencies in patients suffering from conditions associated with such deficiencies.

Generally, the nutritional compositions herein are for use in prevention and treatment in protein-energy malnutrition (PEM). Thus, the nutritional compositions herein are for use in treatment patients with or at risk of protein malnutrition or PEM. Patients can be at risk of PEM in view of a general condition associated with protein wasting, or in view of inadequate dietary protein or protein and calorie intake. Patients can be considered at risk of PEM when consuming diets with inadequate protein intake for prolonged periods of time. For example, patients consuming diets with less than 10 EN % of protein or patients neither meeting neither protein nor calorie requirements.

The nutritional compositions herein are particularly suitable for prevention and treatment of protein-energy deficiencies of elderly patients, pregnant or lactating patients and children (age>1 year). For example for prevention and treatment of protein-energy deficiencies of elderly patients (age>65 yrs), pregnant or lactating patients and children (age>1 year), diagnosed with or at risk of PEM.

In one embodiment, the nutritional compositions of the present disclosure are for use in prevention and treatment of nutrient deficiencies, in particular protein-energy deficiencies associated with frailty and/or sarcopenia in elderly patients (age>65), more preferably hospitalized patients (including patients in nursing homes).

Moreover, the nutritional compositions of the present disclosure are for use in prevention and treatment of nutrient deficiencies associated with neurological disorders, anorexia, chronic obstructive pulmonary disease (COPD).

### Preparation of Nutritional Compositions

The nutritional compositions described herein are obtainable by a process comprising the following steps:

In a first step, water is provided and heated to a temperature of 50-70° C.

In a second step the carbohydrate component—or a fraction thereof—and, optionally, stabiliser, such as MCC, is added.

In a third step the protein component is added.

In a fourth step the lipid component is added, preferably at an elevated temperature of 80-90° C., preferably in combination with a (further) emulsifier and/or stabiliser, such as soy lecithin and/or diacetyl tartric acid of glycerides (e.g. DATEM), and/or (destilled) monoglycerides.

In a fifth step vitamins, minerals and, optionally, the remaining fraction of the carbohydrate component are added. In this step any other powder ingredient may be added. E.g. fibres, aroma, and other additives. At any time before, while or after the fifth step, the mixture is allowed to cool to 55-65° C.

In a sixth step, the pH of the mixture is adjusted to 6-9, preferably 7-8, e.g. 7.

In a seventh step, the mixture is homogenized, e.g. at 60-65° C. at a pressure of 100/50 bar,

In an eighth step, the mixture is sterilized. The eighth step may comprise (8.a) a pre-heating step, (8.b) a heating step and (8.c) an additional homogenization step. For example:
a. in step 8.a the mixture is pre-heated to 70-100° C., preferably 85-95° C., e.g. for 2-5 minutes;
b. in step 8.b the mixture is heated to 130-140° C., preferably to 139-141° C., e.g. for 6-15 seconds; and
c. in step 8c the mixture is homogenized at a pressure of 40-200 bar, preferably step 8.c is carried out at less than 90° C.

In an alternative embodiment, all or a part of the additional ingredients, such as aroma, may be added already between the third and fourth step.

### Dose Unit and Daily Dose

The nutritional compositions herein are typically provided in a dose unit.

A dose unit herein refers to 100-200 mL, preferably 100-150 mL separately provided in package such as a bottle, tetra brick or bag.

Such a dose unit provides 300-500 kcal, preferably 350-450 kcal.

Such a dose unit provides 14-40 g, preferably 15-30 g, more preferably 18-22 g of protein, such as 20 g of protein.

An exemplary dose unit provides 400 kcal and 20 g of protein in a dose unit of 125 mL.

Accordingly, an exemplary daily dose for complete nutrition of 1500-2500 kcal of the nutritional compositions herein may be provided with 3-8 dose units, for example 5 dose units. Accordingly, a daily dose for complete enteral nutrition may be provided by 5 dose units each providing 350-450 kcal,

A typical daily dose for supplemental nutrition of 300-900 kcal of the nutritional compositions herein will be provided by 1-3 dose units. For example by 1-2 dose units each providing 350-450 kcal.

### Embodiments

Embodiment 1: Nutritional composition comprising a protein component, a lipid component, a carbohydrate component, minerals, vitamins and water, wherein the protein component comprises at least two different protein sources; wherein

a) the first protein source is hydrolysed collagen;

b) the lipid component provides at least 30 EN % of the total energy of the composition and

c) the first source of protein represents at least 35 wt %, preferably 45-95 wt % based on the total weight of the protein component

and, preferably the nutritional composition has a pH of 5.5-9.0.

Embodiment 2: Nutritional composition according to embodiment 1 having a caloric density of at least 2 kcal/mL, preferably at least 2.5 kcal/ml, more preferably at least 2.8 kcal/mL most preferably at least 3.0 kcal/mL; the caloric density may have an upper limit of at most 5.0 kcal/mL, preferably at most 4 kcal/ml, most preferred at most 3.8 kcal/mL.

Embodiment 3: Nutritional composition according to embodiment 1 or 2, wherein the hydrolysed collagen has an average molecular weight M_{w} of from 1000 Da to 6000 Da, preferably 1000 to 3000 Da.

Embodiment 4: Nutritional composition according to any of the preceding embodiments, wherein at most 10 wt %, more preferably at most 5 wt % of the collagen hydrolysate has a molecular weight of above 7,500 Da.

Embodiment 5: Nutritional composition according to any of the preceding embodiments, wherein at most 15 wt %, more preferably at most 5 wt % of the collagen hydrolysate has a molecular weight of below 500 Da.

Embodiment 6: Nutritional composition according to any of the preceding embodiments, wherein the collagen hydrolysate is characterized by at least 75 wt %, preferably at least 90 wt % falling into the molecular weight range of 500-7500 Da.

Embodiment 7: Nutritional composition according to any of the preceding embodiments, wherein the hydrolysed collagen has a molecular weight distribution characterized by

| | |
|---|---|
| Mol. weight range | Upper limit |

| | | |
|---|---|---|
| >7500 | Da | 10 wt % |
| >3500-7500 | Da | 35 wt % |
| >1500-3500 | Da | 35 wt % |
| >500-1500 | Da | 50 wt % |
| <500 | Da | 15 wt % |

Embodiment 8: Nutritional composition according to any of the preceding embodiments, wherein the hydrolysed collagen has a molecular weight distribution characterized by

| Mol. weight | | Preferred | | Particularly | | | | | |
|---|---|---|---|---|---|---|---|---|---|
| range | | range | | preferred range | | Example 1 | | Example 2 | |
| >7500 | Da | ≤10 | wt % | ≤5 | wt % | ≤5 | wt % | ≤5 | wt % |
| >3500-7500 | Da | 10-35 | wt % | 10-20 | wt % | 10-20 | wt % | 12-18 | wt % |
| >1500-3500 | Da | 20-35 | wt % | 25-32 | wt % | 25-32 | wt % | 25-31 | wt % |
| >500-1500 | Da | 30-50 | wt % | 40-50 | wt % | 40-50 | wt % | 40-46 | wt % |
| <500 | Da | ≤15 | wt % | ≤15 | wt % | ≤15 | wt % | 5-10 | wt % |

Embodiment 9: Nutritional composition according to any preceding embodiment, wherein the protein component provides at least 15 EN %, preferably at least 18 EN % based on the total energy of the nutritional composition.

Embodiment 10: Nutritional composition according to any of the preceding embodiments comprising at least 14.0 g, preferably at least 15.0 g, more preferably at least 15.5 g, most preferably at least 16 g protein per 100 mL of the nutritional composition; the protein may be present with an upper limit of at most 20 g/100 mL, preferably at most 18 g/100 mL, most preferably at most 17 g/100 mL.

Embodiment 11: Nutritional composition according to any of the preceding embodiments having a protein to water ratio of at least 2.0/10 [g/g], preferably at least 2.5/10 [g/g].

Embodiment 12: Nutritional composition according to any preceding embodiment, wherein the lipid component provides at most 50 EN %, preferably at least 40 EN % based on the total energy of the nutritional composition.

Embodiment 13: Nutritional composition according to any preceding embodiment, wherein the carbohydrate component provides at least 25 EN % based on the total energy of the nutritional composition.

Embodiment 14: Nutritional composition according to any preceding embodiment, wherein

a) the protein component provides 15-25 EN %, preferably 18-22 EN % based on the total energy of the nutritional composition;

b) the lipid component provides 40-50 EN %, preferably 43-47 EN % based on the total energy of the nutritional composition; and

c) the carbohydrate component provides 30-40 EN % preferably 33-37 EN % based on the total energy of the nutritional composition.

Embodiment 15: Nutritional composition according to any preceding embodiment, wherein the second protein source is selected from vegetable proteins, animal proteins other than collagen and mixtures thereof.

Embodiment 16: Nutritional composition according to any of the preceding embodiments wherein the second protein source is selected from soy protein, pea protein, egg white, preferably soy protein.

Embodiment 17: Nutritional composition according to any of the preceding embodiments, wherein the second protein source is selected from milk proteins.

Embodiment 18: Nutritional composition according to any of the preceding embodiments comprising at least 10 wt %, preferably at least 12 wt %, more preferably at least 14 wt % of protein based on the total weight of the composition.

Embodiment 19: Nutritional composition according to any preceding embodiment being an o/w (oil-in-water) emulsion.

Embodiment 20: Nutritional composition according to any of the preceding embodiments having a pH of 5.5-9.0, preferably 7.0-9.0, for example 6-9 or 7-8.

Embodiment 21: Nutritional composition according to any of the preceding embodiments having a viscosity between 50 mPas-900 mPas, preferably 200-750 mPas determined at a shear rate of γ=1 s⁻¹ at 20° C.

Embodiment 22: Nutritional composition according to any preceding embodiment further characterized in being nutritionally complete.

Embodiment 23: Nutritional composition according to any of the preceding embodiments for use in therapy.

Embodiment 24: Use of a protein mixture comprising at least two different sources of protein, wherein the mixture comprises at least 35 wt %, preferably 45-95 wt %, more preferably 70-90 wt %, most preferred 80-85 wt % hydrolysed collagen as a first source of protein based on the total weight of the protein mixture to support or improve homogenization and/or sterilization processing of nutritional emulsions.

Embodiment 25: Use of a protein mixture comprising at least two different sources of protein, wherein the mixture comprises at least 35 wt %, preferably 45-95 wt %, preferably 70-90 wt %, most preferred 80-85 wt % collagen hydrolysate as a first source of protein based on the total weight of the protein mixture to support or improve stability against UHT treatment and/or shelf life of nutritional emulsions and/or to provide nutritional emulsions with adequate viscosity and/or to provide nutritional emulsions of drinkable viscosity.

Embodiment 26: Use according to embodiments 24 or 25, wherein the hydrolysed collagen has an average molecular weight M_{w} of from 1000 Da to 6000 Da, preferably 1000 to 3000 Da.

Embodiment 27: Use according to embodiments 24-26, wherein at most 10 wt %, more preferably at most 5 wt % of the collagen hydrolysate has a molecular weight of above 7,500 Da.

Embodiment 28: Use according to embodiments 24-27, wherein at most 15 wt %, more preferably at most 5 wt % of the collagen hydrolysate has a molecular weight of below 500 Da.

Embodiment 29: Use according to embodiments 24-28, wherein the collagen hydrolysate is characterized by at least 75 wt %, preferably at least 90 wt % falling into the molecular weight range of 500-7500 Da.

### Embodiment 30: Use according to embodiments 24-29, wherein the hydrolysed collagen has a molecular weight distribution characterized by

| | |
|---|---|
| Mol. weight range | Upper limit |

| | | |
|---|---|---|
| >7500 | Da | 10 wt % |
| >3500-7500 | Da | 35 wt % |
| >1500-3500 | Da | 35 wt % |
| >500-1500 | Da | 50 wt % |
| <500 | Da | 15 wt % |

Embodiment 31: Use according to embodiments 24-30, wherein the hydrolysed collagen has a molecular weight distribution characterized by

| Mol. weight | | Preferred | | Particularly | | | | | |
|---|---|---|---|---|---|---|---|---|---|
| range | | range | | preferred range | | Example 1 | | Example 2 | |
| >7500 | Da | ≤10 | wt % | ≤5 | wt % | ≤5 | wt % | ≤5 | wt % |
| >3500-7500 | Da | 10-35 | wt % | 10-20 | wt % | 10-20 | wt % | 12-18 | wt % |
| >1500-3500 | Da | 20-35 | wt % | 25-32 | wt % | 25-32 | wt % | 25-31 | wt % |
| >500-1500 | Da | 30-50 | wt % | 40-50 | wt % | 40-50 | wt % | 40-46 | wt % |
| <500 | Da | ≤15 | wt % | ≤15 | wt % | ≤15 | wt % | 5-10 | wt % |

Embodiment 32: Process for making a nutritional composition comprising a protein component, a lipid component, a carbohydrate component, minerals, vitamins and water, the process comprising the following steps:

a) a first step wherein water is provided and heated to a temperature of 50-70° C.;

b) a second step wherein the carbohydrate component—or a fraction thereof—optionally in combination with an stabilizer, such as MCC, is added;

c) a third step, wherein the protein component is added;

d) a fourth step, wherein the lipid component is added, preferably at an elevated temperature of 80-90° C., preferably in combination with a (further) emulsifier and/or stabiliser, such as soy lecithin, (destilled) monoglycerides or diacetly tartaric acid glycerides;

e) a fifth step wherein vitamins, minerals and, optionally, the remaining fraction of the carbohydrate component are added, optionally, any other powder ingredient may be added in the fifth step, e.g. fibres, aroma, and other additives; at any time before, while or after the fifth step, the mixture is allowed to cool to 55-65° C.

f) a sixth step, wherein the pH of the mixture is adjusted to 6-9, preferably 7-8, e.g. 7.

g) a seventh step, wherein the mixture is homogenized, e.g. at 60-65° C. at a pressure of 100/50 bar,

h) an eighth step, wherein the mixture is sterilized. The eighth step may comprise (8.a) a pre-heating step, (8.b) a heating step and (8.c) an additional homogenization step;.

Embodiment 33: The process of embodiment 32, wherein step 8 is divided into:

a) a step 8.a, wherein the mixture is pre-heated to 70-100° C., preferably 85-95° C., e.g. for 2-5 minutes;

b) a step 8.b, wherein the mixture is heated to 130-140° C., preferably to 139-141° C., e.g. for 6-15 seconds; and

c) a step 8c wherein the mixture is homogenized at a pressure of 40-200 bar, preferably step 8.c is carried out at less than 90° C.

Embodiment 34: The process of embodiments 32 or 33, wherein the protein component comprises at least two different protein sources; wherein the first protein source is hydrolysed collagen.

Embodiment 35: The process of embodiment 34, wherein the hydrolysed collagen has an average molecular M_{w} of from 1000 Da to 6000 Da, preferably 1000 to 3000 Da.

Embodiment 36: The process of embodiments 34 or 35, wherein at most 10 wt %, more preferably at most 5 wt % of the collagen hydrolysate has a molecular weight of above 7,500 Da.

Embodiment 37: The process of embodiments 34-36, wherein at most 15 wt %, more preferably at most 5 wt % of the collagen hydrolysate has a molecular weight of below 500 Da.

Embodiment 38: The process of embodiments 34-37, wherein the collagen hydrolysate is characterized by at least 75 wt %, preferably at least 90 wt % falling into the molecular weight range of 500-7500 Da.

Embodiment 39: The process of embodiments 34-38, wherein the hydrolysed collagen has a molecular weight distribution characterized by

| | |
|---|---|
| Mol. weight range | Upper limit |

| | | |
|---|---|---|
| >7500 | Da | 10 wt % |
| >3500-7500 | Da | 35 wt % |
| >1500-3500 | Da | 35 wt % |
| >500-1500 | Da | 50 wt % |
| <500 | Da | 15 wt % |

Embodiment 40: The process of embodiments 34-39, wherein the hydrolysed collagen has a molecular weight distribution characterized by

| Mol. weight | | Preferred | | Particularly | | | | | |
|---|---|---|---|---|---|---|---|---|---|
| range | | range | | preferred range | | Example 1 | | Example 2 | |
| >7500 | Da | ≤10 | wt % | ≤5 | wt % | ≤5 | wt % | ≤5 | wt % |
| >3500-7500 | Da | 10-35 | wt % | 10-20 | wt % | 10-20 | wt % | 12-18 | wt % |
| >1500-3500 | Da | 20-35 | wt % | 25-32 | wt % | 25-32 | wt % | 25-31 | wt % |
| >500-1500 | Da | 30-50 | wt % | 40-50 | wt % | 40-50 | wt % | 40-46 | wt % |
| <500 | Da | ≤15 | wt % | ≤15 | wt % | ≤15 | wt % | 5-10 | wt % |

Embodiment 41: Nutritional composition obtainable by the process of embodiment 32-40.

Embodiment 42: Nutritional composition according to any of embodiments 1-22 obtainable by the process of claims 32-40.

Embodiment 43: Dose unit comprising the nutritional composition of any of embodiments 1-22, 41 or 42.

Embodiment 44: Dose unit according to embodiment 43 providing 300-500 kcal, preferably 350-450 kcal.

Embodiment 45: Dose unit according to embodiment 43 or 44 comprising 100-200 mL, preferably 100-150 mL of the nutritional composition, preferably in package such as a bottle, tetra brick or bag.

Embodiment 46: Dose unit according to embodiments 43-45 comprising 15-30 g, preferably 18-22 g of protein.

Embodiment 47: Dose unit according to embodiment 43-46 providing 400kcal and 20 g of protein in 125 mL.

Embodiment 48: Dosage regime for use in (nutritional) therapy of patients requiring fluid restriction wherein a daily dose for complete nutrition is provided with 3-8 dose units according to embodiments 43-47, for example with 5 dose units each providing 350-450 kcal.

Embodiment 49: Dosage regime of embodiment 48, wherein the daily dose for complete nutrition refers to 1500-2500 kcal.

Embodiment 50: Dosage regime for use in (nutritional) therapy of patients requiring fluid restriction wherein a daily dose for supplemental nutrition is provided with 1-3 dose units, preferably 1-2 dose units according to embodiments 43-47.

Embodiment 51: Dosage regime of embodiment 50, wherein the daily dose for supplemental nutrition refers to 300-900 kcal, for example with 1 or 2 dose units each providing 350-450 kcal.

Embodiment 52: Dosage regime according to embodiments 48-51 for use in treatment or prevention of malnutrition in patients requiring fluid restriction.

Embodiment 53: Nutritional composition according to embodiments 1-22 for use in therapy, wherein the composition is used in treatment or prevention of protein malnutrition or protein energy malnutrition.

Embodiment 54: Nutritional composition according to embodiments 1-22 for use in therapy, wherein the nutritional composition is used in prevention and treatment of nutrient deficiencies associated with malnutrition.

Embodiment 55: Nutritional composition according to embodiments 1-22 for use in therapy, wherein the nutritional composition is used in prevention and treatment of nutrient deficiencies associated with therapeutic fluid restriction, for example in patients requiring therapeutic fluid restriction selected from dialysis patients, patients with cardiac insufficiency and patients with liver insufficiency.

Embodiment 56: Nutritional composition according to embodiments 1-22 for use in therapy, wherein the nutritional composition is used in prevention and treatment of nutrient deficiencies associated with (chronic) wasting diseases such as catabolism, cancer cachexia, HIV, chronic wounds or sarcopenia.

Embodiment 57: Nutritional composition according to embodiments 1-22 for use in therapy, wherein the nutritional composition is used in prevention and treatment of nutrient deficiencies in any condition associated with increased protein needs or overall energy, preferably both, for example for use in prevention and treatment of nutrient deficiencies in gastrointestinal conditions associated with protein and/or energy wasting, for use in prevention and treatment of nutrient deficiencies of surgical patients, for use in prevention and treatment of nutrient deficiencies associated with wounds and pressure ulcers or for use in prevention and treatment of nutrient deficiencies associated with cancers.

Embodiment 58: Nutritional composition according to embodiments 1-22 for use in therapy, wherein the nutritional composition is used in prevention and treatment of protein-energy deficiencies in patients suffering from conditions associated with such deficiencies.

Embodiment 59: Nutritional composition according to embodiments 1-22 for use in therapy, wherein the nutritional composition is used in prevention and treatment of protein-energy malnutrition (PEM) preferably for use in treatment of patients with or at risk of protein malnutrition or PEM.

Embodiment 60: Nutritional composition according to embodiments 1-22 for use in therapy, wherein the nutritional composition is used in prevention and treatment of protein-energy deficiencies of elderly patients (age>65), pregnant or lactating patients and children (age>1 year), preferably for prevention and treatment of protein-energy deficiencies of elderly patients (age>65), pregnant or lactating patients and children (age>1 year) who have been diagnosed with or at risk of PEM.

Embodiment 61: Nutritional composition according to embodiments 1-22 for use in therapy, wherein the nutritional composition is used in prevention and treatment of nutrient deficiencies, preferably protein-energy deficiencies, associated with frailty and/or sarcopenia in elderly patients (age>65), more preferably hospitalized patients (including patients in nursing homes).

Embodiment 62: Nutritional composition according to embodiments 1-22 for use in nutritional therapy.

Embodiment 63: Dosage regime for use in (nutritional) therapy according to embodiments 58-61, wherein a daily dose for complete nutrition is provided with 3-8 dose units according to embodiments 43-47, for example with 5 dose units each providing 350-450 kcal.

Embodiment 64: Dosage regime of embodiment 63, wherein the daily dose for complete nutrition refers to 1500-2500 kcal.

Embodiment 65: Dosage regime for use in (nutritional) therapy embodiments 58-61, wherein a daily dose for supplemental nutrition is provided with 1-3 dose units, preferably 1-2 dose units according to embodiments 43-47, for example with 1 or 2 dose units each providing 350-450 kcal.

Embodiment 66: Dosage regime of embodiment 65, wherein the daily dose for supplemental nutrition refers to 300-900 kcal, for example with 1 or 2 dose units each providing 350-450 kcal.

Embodiment 67: Dosage regime of embodiments 48-52 or 63-66, wherein each dose unit provides 15-30 g, preferably 18-22 g of protein.

It is to be understood that the present disclosure also relates to a method of treating the deficiencies and conditions described in the section “use in therapy” comprising the step of administering the nutritional compositions or dose units described herein to a human subject. In particular, the present disclosure relates to such methods comprising the step of administering the nutritional compositions or dose units according to the dosage regime described herein.

It is further to be understood that the present disclosure also relates to the use of the nutritional compositions herein in the manufacture of a therapeutic composition or medicament for use in therapy or treatment as described herein. In particular, such therapy may comprise administering the nutritional compositions, preferably as dose units, according to the dosage regime described herein.

### EXEMPLES

Nutritional compositions according to table 1 were prepared. After mixing, a homogenization step was conducted.

Example 1 is a standard nutritional composition already high in calories with a protein component solely based on milk protein. While being stable and homogeneous at 2.0 kcal and 20 EN % protein (Example 1), it was not possible to obtain a sufficiently homogeneous composition when increasing caloric density (Example 2).

Surprisingly, increasing caloric density was possible by adding collagen hydrolysate in sufficient amounts to milk protein (Example 4 and Example 5). Example 3 is a comparative example with insufficient amount of collagen hydrolysate.

Example 4 further shows that with inclusion of sufficient collagen hydrolysate very high energy densities of protein can be reached in a formula that provides high caloric density (Example 4: 2.6 kcal) or very high caloric density (Example 5: 3.2 kcal/mL).

The inventors further observed that fouling during heat treatment could be reduced/prevented when increasing wt % of collagen hydrolysate in the protein component (Example 5).

With the nutritional composition, a drinkable viscosity of nutritionally complete compositions can be provided inspite high protein and energy content.

The inventors further observed that bitterness of the product could be reduced by increasing wt % of collagen hydrolysate in the protein component. Therefore, the nutritional composition will result in a better patient compliance.

### Trial

The nutritional composition according to Example 5 (test drink) is given to subjects with an indication for supplemental nutrition of approx. 400 kcal per day. Subjects belong to the age group of 65 and older. Age, sex, height and BMI are recorded. The dosage is one bottle of 125 ml per day for seven consecutive days. One such bottle provides 400 kcal, including 20 g protein (20 EN %), 20 g fat (45 EN %) and 35 g carbohydrates (35 EN %).

Adverse effects are documented. Gastrointestinal tolerance parameters are documented. Moreover, (protein) energy intake, compliance (in particular time until full consumption of the test drink and/or amount consumed within 1 hour), activity level and palatability are documented.

### Methods

### Determination of Molecular Weight of the Hydrolysed Collagen by GPC/HPLC

Equipment: GPV/HPLC with UV detector operating at 214 nm. Column: TSK 2000 SW XL (Toshoh Biosience GmbH). Isocratic elution using 400 mmol/l sodium phosphate buffer (pH 5.3). Calibration by means of well-defined Type I-collagen fragments (FILK, Freiburg, Germany). The collagen hydrolysate used in the examples had an average molecular weight of 2 kDa. In general, the skilled person is well aware of molecular mass determination via GPC. Another suitable method for determining the M_{w} of small macromolecules such as the hydrolysates described herein is MALDI-MS.

TABLE 1
Exempels

| | Example 1 | Example 2 | Example 3 | Example 4 | Example 5 |
|---|---|---|---|---|---|
| Protein source 1 | −/− | −/− | Collagen hydrolysate | Collagen hydrolysate | Collagen hydrolysate |
| | | | 28.6% | 49% | 80% |
| Protein Source 2 | Milk protein 100% | Milk protein 100% | Milk protein 71.4% | Milk protein 51% | Milk protein 20% |
| | (Refit 54% MPC-80 | (Refit ® MCI 88) | (Refit 38.8% MPC-80 | (Refit 28% MPC-80 | (Refit 10.8%/MPC-80 |
| | 46%) | | 32.6%) | 23%) | 9.2%) |
| Protein | 20 EN % | 20 EN % | 26 EN % | 20 EN % | 20 EN % |
| CHO | 35 EN % | 30 EN % | 33 EN % | 35 EN % | 35 EN % |
| Fat | 45 EN % | 50 EN % | 41 EN % | 45 EN % | 45 EN % |
| Caloric density | 2.0 kcal | 3.2 kcal/L | 3.2 kcal/L | 2.6 kcal | 3.2 kcal/L |
| FSMP balanced * | yes | No | No | yes | yes |
| Homogenization method | 100/50 bar | 100/50 bar | 100/50 bar | 100/50 bar | 100/50 bar |
| (1^{st} step/2^{nd} step) | | | | | |
| Homogenization/heat | homogeneous | Not possible to | Possible to homogenize. | Possible to homogenize. | Possible to homogenize. |
| treatment | | homogenize. | However the matrix was | In case of (optional) | Compared to example 4: |
| | | Consequently, no heat | not stable during | subsequent UHT | Improved stability |
| | | treatment was applied. | Autoclave Heat | sterilization, fouling in | against subsequent |
| | | | Treatment, the product | the line was observed. | UHT sterilization, no |
| | | | became a curdly mass | | fouling. |
| | | | with expelled water. | | Lower viscosity |
| | | | | | Improved shelf life |
| | | | | | (i.e. at least ? months) |

* Nutritionally complete in vitamins and minerals

TABLE 2
Vitamins

| | Minimum per 100 kcal | Maximum per 100 kcal |
|---|---|---|
| Vitamin A (μg RE) | 35 | 180 |
| Vitamin D (μg) | 0.5 | 3 |
| Vitamin K (μg) | 3.5 | 20 |
| Vitamin C (mg) | 2.2 | 22 |
| Thiamin (mg) | 0.06 | 0.5 |
| Riboflavin (mg) | 0.08 | 0.5 |
| Vitamin B6 (mg) | 0.08 | 0.5 |
| Niacin (mg EN) | 0.9 | 3 |
| Folic acid (μg) | 10 | 50 |
| Vitamin B12 (μg) | 0.07 | 0.7 |
| Pantothenic acid (mg) | 0.15 | 1.5 |
| Biotin (μg) | 0.75 | 7.5 |
| Vitamin E (mg α-TE) | 0.5 | 3 |

TABLE 3
Minerals

| | Minimum per 100 kcal | Maximum per 100 kcal |
|---|---|---|
| Sodium (mg) | 30 | 175 |
| Chloride (mg) | 30 | 175 |
| Potassium (mg) | 80 | 295 |
| Calcium (mg) | 35 | 250 |
| Phosphorus (mg) | 30 | 80 |
| Magnesium (mg) | 7.5 | 25 |
| Iron (mg) | 0.5 | 2.0 |
| Zinc (mg) | 0.5 | 1.5 |
| Copper (μg) | 60 | 500 |
| Iodine (μg) | 6.5 | 35 |
| Selenium (μg) | 2.5 | 10 |
| Manganese (mg) | 0.05 | 0.5 |
| Chromium (μg) | 1.25 | 15 |
| Molybdenum (μg) | 3.5 | 18 |
| Flouride (mg) | — | 0.2 |

1. Nutritional composition comprising a protein component, a lipid component, a carbohydrate component, minerals, vitamins and water, wherein the protein component comprises at least two different protein sources; wherein the nutritional composition has a pH in the range of 5.5-9.0 and wherein
a) the first protein source is hydrolysed collagen;
b) the lipid component provides at least 30 EN % of the total energy of the composition and
c) the first source of protein represents 35-95 wt % based on the total weight of the protein component.

2. Nutritional composition according to claim 1 having a caloric density of at least 2 kcal/mL.

3. Nutritional composition according to claim 1 or 2 wherein the first protein source represents at least 45 wt % based on the total weight of the protein component.

4. Nutritional composition according to any preceding claim, wherein the protein component provides at least 15 EN % based on the total energy of the nutritional composition.

5. Nutritional composition according to any of the preceding claims comprising at least 14 g protein per 100 mL product.

6. Nutritional composition according to any of the preceding claims having a protein to water ratio of at least 2.0/10 [g/g].

7. Nutritional composition according to any preceding claim, wherein the lipid component provides at most 50 EN % based on the total energy of the nutritional composition.

8. Nutritional composition according to any preceding claim, wherein the carbohydrate component provides at least 25 EN % based on the total energy of the nutritional composition.

9. Nutritional composition according to any preceding claim, wherein
a) the protein component provides 15-25 EN % based on the total energy of the nutritional composition;
b) the lipid component provides 40-50 EN % based on the total energy of the nutritional composition; and
c) the carbohydrate component provides 30-40 EN % based on the total energy of the nutritional composition.

10. Nutritional composition according to any preceding claim, wherein the second protein source is selected from vegetable proteins, animal proteins other than collagen and mixtures thereof.

11. Nutritional composition according to any of the preceding claims wherein the second protein source is selected from soy protein, pea protein and egg white.

12. Nutritional composition according to any of the preceding claims, wherein the second protein source is selected from milk proteins.

13. Nutritional composition according to any of the preceding claims comprising at least 10 wt % of protein based on the total weight of the composition.

14. Nutritional composition according to any preceding claim being an o/w emulsion.

15. Nutritional composition according to any of the preceding claims having a pH of 7-9.

16. Nutritional composition according to any preceding claim further characterized in being nutritionally complete.

17. Nutritional composition according to any of the preceding claims for use in therapy.

18. Nutritional composition according to claim 17 for use in prevention and treatment of malnutrition.

19. Use of a protein mixture comprising at least two different sources of protein, wherein the mixture comprises at least 35 wt %, preferably 45-95 wt %, more preferably 70-90 wt %, most preferred 80-85 wt % collagen hydrolysate as a first source of protein based on the total weight of the protein mixture to support or improve homogenization of nutritional emulsions.

20. Use of a protein mixture comprising at least two different sources of protein, wherein the mixture comprises at least 35 wt %, preferably 45-95 wt %, more preferably 70-90 wt %, most preferred 80-85 wt % collagen hydrolysate as a first source of protein based on the total weight of the protein mixture to support or improve stability against UHT treatment and/or shelf life of nutritional emulsions.

21. Process for making a nutritional composition comprising a protein component comprising collagen hydrolysate, a lipid component, a carbohydrate component, minerals, vitamins and water, the process comprising the following steps:
i. a first step wherein water is provided and heated to a temperature of 50-70° C.;
ii. a second step wherein the carbohydrate component—or a fraction thereof—and, optionally, an stabilizer, such as MCC, is added;
iii. a third step, wherein the protein component is added;
iv. a fourth step, wherein the lipid component is added, preferably at an elevated temperature of 80-90° C., preferably in combination with a (further) emulsifier and/or stabiliser, such as soy lecithin and/or diacetyl tartric acid of glycerides (e.g. DATEM), and/or (destilled) monoglycerides.;
v. a fifth step wherein vitamins, minerals and, optionally, the remaining fraction of the carbohydrate component are added, optionally, any other powder ingredient may be added in the fifth step, e.g. fibres, aroma, and other additives. At any time before, while or after the fifth step, the mixture is allowed to cool to 55-65° C.
vi. a sixth step, wherein the pH of the mixture is adjusted to 6-9, preferably 7-8, e.g. 7.
vii. a seventh step, wherein the mixture is homogenized, e.g. at 60-65° C. at a pressure of 100/50 bar,
viii. an eighth step, wherein the mixture is sterilized. The eighth step may comprise (8.a) a pre-heating step, (8.b) a heating step and (8.c) an additional homogenization step; and

22. The process of claim 21, wherein step 8 is divided into:
a) a step 8.a, wherein the mixture is pre-heated to 70-100° C., preferably 85-95° C., e.g. for 2-5 minutes;
b) a step 8.b, wherein the mixture is heated to 130-140° C., preferably to 139-141° C., e.g. for 6-15 seconds; and
c) a step 8c wherein the mixture is homogenized at a pressure of 40-200 bar, preferably step 8.c is carried out at less than 90° C.

23. Nutritional composition obtainable by the process of claim 21 or 22.

24. Nutritional composition according to any of claims 1-16 obtainable by the process of claim 21 or 22.
